(19) United States
(12) Patent Application Publication
(10) Pub. No.: US 2018/0104503 A1
(43) Pub. Date: Apr. 19, 2018

(54) COLD ATMOSPHERIC PLASMA TREATMENT OF ACTINIC KERATOSIS AND NON-MELANOMA SKIN CANCER

(71) Applicant: Michelle Wirtz, Düsseldorf (DE)

(72) Inventor: Michelle Wirtz, Düsseldorf (DE)

(21) Appl. No.: 15/640,862

(22) Filed: Jul. 3, 2017

Related U.S. Application Data
(60) Provisional application No. 62/408,765, filed on Oct. 15, 2016.

(51) Int. Cl. A61N 1/44; A61K 31/196; A61K 31/22; A61K 31/513; A61K 31/4745; A61K 41/00

(52) U.S. Cl. CPC A61N 1/44; A61K 31/196; A61K 41/0061; A61K 31/513; A61K 31/4745; A61K 31/22

(57) ABSTRACT

The present invention relates to a new method of treating patients with the skin conditions including actinic keratosis and non-melanoma skin cancer using Cold Atmospheric Plasma (CAP).

### CROSS REFERENCE TO RELATED APPLICATIONS

This application claims priority from U.S. Provisional application No. 62/408,765, filed on Oct. 15, 2016.

### BACKGROUND OF THE INVENTION

### Field of the Invention

The invention relates to the field of Cold Atmospheric Plasma (CAP) treatment of Actinic Keratosis and Non-Melanoma Skin Cancer.

### Description of Related Art

“Hot” (thermic) Atmospheric Plasma is well established for ablation or coagulation, but because of the temperature (>10,000° Celsius) it is not possible to use it on physiological surfaces, including skin. Since some years “Cold” (non-thermic) Atmospheric Plasma (CAP) can be generated and used for such surfaces. CAP is a (partially or fully) ionized gas composed of multiple chemically active species, such as ions, electrons, photons, reactive oxygen and nitrogen species, and UV light. The active species of CAP are capable of inducing physical phenomena and chemical reactions on biological surfaces upon application. CAP has demonstrated significant anti-microbial efficacy in clinical trials. The common use of CAP in the medical field is e.g. surgeries, endoscopic procedures, to deactivate pathogens, stop bleeding, and actively promote wound healing by stimulation of cell proliferation of e.g. basal keratinocytes or fibroblasts. Despite this pro-proliferative effect of CAP, recent pre-clinical observations suggested an anti-tumoral potential of CAP in different malignant tumors, for example melanoma, glioma and colorectal carcinoma cells, while the biological effects and the underlying mode of action still remain unclear. Hitherto, harmful clinical effects have not been reported and in vitro studies did not show increased genotoxicity in cultured cells after repetitive argon plasma treatment. So far, there is no evidence for harmful clinical effects by CAP and in vitro studies could not find an increase of genotoxicity in cultured cells after treatment with plasma, such as argon plasma, up to 180 seconds (Wende et al., Mutat Res Genet Toxicol Environ Mutagen 2016; 798-799: 48-54; Maisch T, Bosserhoff A K, Unger P et al. Investigation of toxicity and mutagenicity of cold atmospheric argon plasma. Environ Mol Mutagen 2017; 58: 172-7).

In the presently described invention, it has been surprisingly found that CAP can be used to treat Actinic Keratosis and Non-Melanoma Skin Cancer with a high rate of success and no or essentially no adverse side effects.

### SUMMARY OF THE INVENTION

Actinic keratosis (AK) is the most frequent epidermal neoplasia that occurs in fair-skinned people with increased cumulative exposure to UV-radiation. In 2005, AK affected more than 58 million people in the USA with a reported prevalence of 11-26% according to recent reports. AK clinically appears as erythematous, hyperkeratotic, scaly or crusty macule, papule and plaque or, in its disseminated form, as field cancerization in chronically sun-exposed skin areas. Histologically, AK is characterized by intraepidermal keratinocytic dysplasia, which can transform into invasive squamous cell carcinoma (SCC) in up to 20% of the AK lesions within 10-25 years. SCC is a common form of Non-Melanoma Skin Cancer (NMSC). NMSC is the most common cancer occurring in people with fair skin and is a rising problem for health care services because it is more common than all other forms of cancer combined—with increasing incidence rates.

AK comprises pre-cancerous skin lesions, therefore selective and efficient therapy is essential to prevent neoplastic transformation. Depending on the clinical manifestation of AK and individual patient's characteristics, various therapy options are available and recommended in dermatological guidelines. Known therapies of AK used to prevent progress to NMSC include: topical treatments, such as self-application by the patient of cytostatic, immune-modifying or antineoplastic substances (e.g. diclofenac (sodium), ingenol mebutate, 5-fluorouracil, imiquimod (e.g. 3.75 or 5%) over a long specified period; photodynamic therapy, defined as selective destruction of tumor cells through application of photosensitization substance (e.g. aminolevulinic acid or methyl aminolevulinate (as hydrochloride)) followed by radiation with a special device (or daylight); and invasive treatments, such as cryotherapy, curettage, ablative laser or removal by surgical operation. All these known treatments are subject to side effects.

Topical treatments may produce serious side effects, including paresthesia, pain, toxic agranulocytosis, edema, bleeding, renal insufficiency, rash, local reactions/infections, chills, fever, arthralgia, nausea, headache, diarrhea, depression, alopecia, allergic reactions up to Stevens-Johnson-Syndrome.

Photodynamic therapy side effects include massive neuropathic pain (during therapy time), inflammatory pain, visual impairment up to blindness, edema, dermatitis solaris, hyperpigmentation, crust and scars. Side effects of invasive treatments include allergic reaction to anesthetics, pain, infections, bleeding, blistering, necrosis, wound healing disorder, hypo- or hyperpigmentation, scars and deforming cosmetic results.

There is a need for a non-invasive, simple and effective treatment for AK and NMSC that also has minimal side effects. It has now been surprisingly found that CAP can be used to treat AK and probably NMSC with a high rate of success and no or essentially no adverse side effects.

### BRIEF DESCRIPTION OF FIGURES

The patent or application file contains at least one drawing executed in color. Copies of this patent or patent application publication with color drawing(s) will be provided by the Office upon request and payment of the necessary fee. The FIGURE provides clinical images (patients 01, 02, 04, 06-2) showing actinic keratosis prior to (1^{st} column), during (after three CAP treatments, 2^{nd} column) and after seven CAP treatments (3^{rd} column).

### DETAILED DESCRIPTION

A novel method of treating AK and/or NMSC is provided which includes applying CAP to the affected area by using any CE-certified CAP device (e.g. Adtec SteriPlas by Adtec Plasma technology). The cold atmospheric plasma may be an argon containing plasma, more particularly an argon containing plasma generated by microwaves. The plasma may be generated by an ionization chamber as described in WO 2007/031250, which is incorporated herein by reference. In the ionization chamber, argon gas is bombarded with electrons emitted from multiple hot electric filaments. The resulting plasma ions mix with air creating reactive agents to generate a wide uniform treatment field that is capable of treating larger tissue areas of about 4-900 cm². Unlike air-based plasmas that vary with temperature, pressure, and location, neutral argon plasma is a consistent and controllable energized gas with predictable active agents and constituents that include reactive oxygen and nitrogen species, OH radicals, ions, electrons, photons, UV light and activated argon species. Particularly, the cold atmospheric plasma has a comparatively low temperature, which is preferably below 100° C., 75° C. or even 50° C., more particularly 20° C. The cold atmospheric plasma particularly has a temperature of 20-100° C., more preferably 20-80° C., particularly when measured at a distance of 20-100 mm from the ionization electrode.

Particularly, the device is configured such that the distance between the patients' affected areas to be treated with CAP and the ionization electrodes is controlled to be 2.5-30 cm, preferably 3-20 cm, most preferably 3.5-15 cm.

No tools, wound dressings, ointments or other procedures are necessary. Preferably the method of treating AK (and/or NMSC) includes regular CAP treatment of the AK affected area, similar to the treatment of chronic wounds. According to the novel method, a patient affected with AK (and/or NMSC) is treated by exposing the affected area to cold atmospheric plasma for a time sufficient to induce an antitumoral response sufficient to destroy cancerous cells. The treatment is repeated until the AK (or NMSC) is sufficiently eliminated and clearance is achieved. Clearance means reduction of target lesions (there are less lesions after the treatment than at the beginning of the treatment (t=0) and/or improvement of the Olson grade (that is, the number of lesions are the same after the treatment, however the severity of the lesions is improved). Ideally clinically satisfactory clearance is reached, including complete clearance, that is no lesions after treatment. In an embodiment of the method, for example, the duration of the application is preferably at least about two minutes, and is preferably repeated once or twice a week for several applications until the desired clearance is achieved.

In a preferred embodiment, the patient's affected area is exposed to the cold (argon) plasma according to the invention for 1-6 sessions a week, preferably two sessions a week. It is preferred that there is not more than one session a day. Thus, in another aspect, the patient's affected area is exposed to cold atmospheric plasma for one session a day, one session every two days, one session every four days, one session every five days or one session every six days.

In one aspect, the affected area is exposed to cold atmospheric plasma for 10-300 seconds per session, preferably 20-150 seconds per session, and most preferably 70-140 seconds.

In most cases, clearance is achieved after 5-30, preferably 5-20 sessions in total.

It was shown that the treatment according to the invention is particularly effective in patients who are fair-skinned (Fitzpatrick skin types I, II or III). The method of the invention is particularly effective in patients suffering from therapy-refractory actinic keratosis. Particularly patients suffering from actinic keratosis at an Olsen grade I, II or III, more particularly II or III, even more particularly II can effectively be treated by the method of the invention. The method according to the invention is also effective in patients suffering from field cancerization or untreated actinic keratosis.

In another aspect, the method of the invention was shown to be effective in patients that were previously treated with diclofenac, particularly diclofenac in hyaluronic acid, ingenol mebutate, 5-fluorouracil and/or with imiquimod or photodynamic therapy, preferably using aminolevulinic acid, which treatment, however, did not result in a satisfactory clearance.

The method of the invention may comprise a simultaneous or a consecutive treatment with an active agent against actinic keratosis, particularly with at least one of diclofenac (sodium), ingenol mebutate, 5-fluorouracil optionally together with salicylic acid, (methyl)aminolevulinic acid or salts thereof or imiquimod or photodynamic therapy, preferably using aminolevulinic acid or (methyl)aminolevulinate (e.g. as hydrochloride).

In another aspect, the method of the invention is particularly effective in patients who are not simultaneously treated with an additional active agent against actinic keratosis during the treatment according to the invention.

Because AKs or NMSCs generally do not disappear immediately after CAP treatment, but the treatment induces an antitumoral response, a follow-up assessment should be conducted to ensure that the treatment has achieved clinically satisfactory clearance. Depending on the patient and condition, the application time, and repetition interval and number of applications needed to achieve the desired clearance may vary.

The method of treatment of the invention has negligible side effects. While all established therapies cause local painful inflammatory or immune-modulating reactions—some even systemic side effects—CAP is completely painless and leaves healthy tissue unaffected since it is specific for tumor cells.

It was also shown that the method according to the present invention results in a continuous decline of erythema, scaling, crusts and thickness thereof, lesions and in a reduction of the total lesion number. Another interesting effect of CAP that was found is a potentially rejuvenating effect on the skin tone, lentigines, roughness and texture similar to that reported for other dermal remodeling physical procedures such as photodynamic therapy (PDT). A refined complexion, a reduction of hyper- or mottled pigmentation, and a reduction of hyperkeratosis and a palpatory firmer skin was observed in a subgroup of patients.

Unless defined otherwise, all technical and scientific terms used herein have the meaning commonly understood by a person skilled in the art to which this invention belongs. The meaning and scope of terms should be clear; however, in the event of any latent ambiguity, definitions provided herein take precedent over any dictionary or extrinsic definition. In this application, the use of “or” means “and/or” unless stated otherwise. Furthermore, the use of the term “including” as well as other forms of same such as “includes” and “included” is not limiting. With reference to the use of the words “comprise” or “comprises” or “comprising” in this patent application, Applicants note that unless the context requires otherwise, those words are used on the basis and clear understanding that they are to be interpreted inclusively, rather than exclusively, and that Applicants intend each of those words to be so interpreted in construing the patent application, including the claims.

As used in the specification and claims, the singular form “a,” “an,” and “the” include plural references unless the context clearly dictates otherwise.

In this disclosure, “comprise,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Where a range of values is provided, it is understood that each intervening value, to the tenth of the unit of the lower limit, unless the context clearly dictates otherwise, between the upper and lower limit of that range and any other stated or intervening value in that stated range, is encompassed within the invention. The upper and lower limits of these ranges may independently be included in the smaller ranges and are also encompassed within the invention, subject to any specifically excluded limit in the stated range. Where the stated range includes one or both of the limits, ranges excluding either or both of those included limits are also included in the invention.

The following non-limiting examples are provided to further illustrate the present invention.

### Examples

### Example 1. Clinical Trial in Patients with Actinic Keratosis

Seven patients (2 females, 5 males) with clinically diagnosed AK in their 50s to 80s were treated. All of them were fair-skinned with Fitzpatrick skin types II or III. All suffered from multiple, therapy-refractory AK or field-cancerization, mostly due to a history of extensive UV exposure (Table 1). One patient was under immune suppressive treatment with MTX 10 mg weekly because of psoriasis vulgaris et arthropathica.

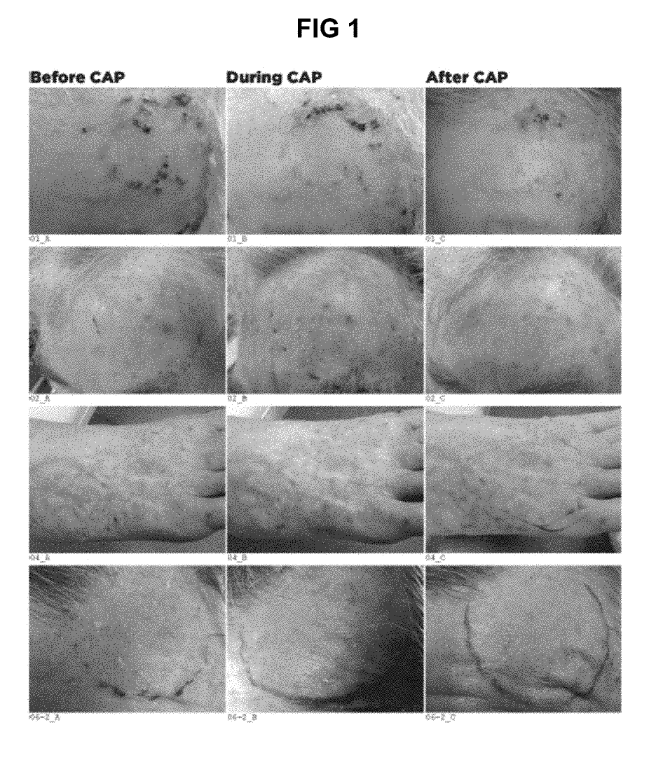

In each patient, a circular area of 19.64 cm², containing multiple therapy-refractory AK was defined, photo-documented (FIG. 1, 1^{st} column), and the lesions were clinically classified according to Olsen grading: I slightly palpable, better felt than seen; II moderately thick, easily felt and seen; III very thick and hyperkeratotic. Seven CAP treatments were performed with a microwave-driven argon plasma jet (Adtec SteriPlas™ argon plasma (power consumption 1.5 kVA)) in each patient (twice a week, 120 seconds per treatment) after detailed explanation and obtaining informed consent.

During the CAP treatment period, no other concomitant therapy for AK was applied except standard physical UV protection procedures. Photo-documentation of the defined areas was done during and after seven CAP treatments (FIG. 1, 2^{nd} and 3^{rd} columns).

A continuous decline of clinical AK characteristics such as erythema, scaling, crusts and thickness could be found in all eight treated areas (Table 2). In six areas, this resulted in clinical downgrading of the lesions according to Olsen grading and to a reduction of the total lesion number (Table 2). The other two treated areas showed limited total change from baseline, but a clinical Olsen downgrading of the existing lesions. A refined complexion with a discrete reduction of hyper- or mottled pigmentation seemed to occur in patients 3 and 7, a slightly firmer skin in patient 6 (site no. 1, tibia) and a reduction of hyperkeratosis in patients 3 and 4. No adverse events were reported and the treatment was well tolerated by all patients.

Discussion of Clinical Results:

All patients treated in this case series showed promising responses after seven applications of CAP. Even though there are a variety of therapies for AK, CAP could become an important alternative for treatment because of its lack of side effects. In the study, no patient reported side effects, particularly no local pain or inflammation. Current physical (e.g. photodynamic therapy (PDT) or cryotherapy) and topical (e.g. imiquimod or ingenol mebutate) treatments for AK usually provoke local skin reactions such as erythema, flaking, scaling and crusting. Even for rather well tolerated topical agents, such as diclofenac 3% in 2.5% hyaluronic acid, dermatitis is a commonly reported side effect. Moreover, many local AK therapies such as fluorouracil or diclofenac cause photosensitivity, which limits their use in the summer.

The absence of side effects after CAP was, however, repeatedly documented also from numerous applications in chronic wound management where CAP is already frequently applied. Most likely, this allows CAP application also for larger body areas without a limit in the maximum treatment area, making it an attractive option especially for patients with extensively chronically sun-damaged skin or even immune-compromised patients. Other common topical therapies, such as 5% imiquimod, are limited to 25 cm².

Another interesting effect of CAP observed in a subgroup of patients is a potentially rejuvenating effect on the skin tone, lentigines, roughness and texture similar to that reported for other dermal remodeling physical procedures such as photodynamic therapy (PDT). A refined complexion, a reduction of mottled pigmentation and a firmer skin were observed in patients 3, 4, 6, and 7. The biological mode of action regarding the anti-tumorigenic potential of CAP remains unclear. Effects on the cell cycle and induction of senescence, apoptosis, and necrosis by reactive nitrogen and oxygen species have been discussed. Interestingly, the tumor-selectivity and apoptotic effect of CAP are especially high in p53-mutated cancer cells. Dysregulation of the p53 pathway plays an important role in inducing clonal keratinocytic expansion in AK and SCC. Since human papilloma viruses are considered co-carcinogenic in AK and SCC development and infected keratinocytes frequently express HPV oncoproteins, virocide properties of CAP might work synergistically.

In conclusion, even though there exists a variety of AK therapies, CAP might represent a novel and safe treatment because of its lack of side effects.

TABLE 1
Patient overview (n = 7)

| | | | | | | | Cold |
|---|---|---|---|---|---|---|---|
| | | | | | | | atmospheric |
| | | | | UV-Exposure | | Prior actinic | plasma |
| | | | Fitzpatrick | and special | Prior history of | keratosis | treatment |
| Patient | Sex | Age | skin type | features | skin cancer | treatments | area |
| 01 | m | 82 | II | increased | squamous cell | Diclofenac | left forehead |
| | | | | exposure | carcinoma (right | 3% to 2.5% | (cf. FIG. 1/01) |
| | | | | childhood | shoulder, temporal | hyaluronic acid | |
| | | | | | right, left cheek | | |
| | | | | | lateral, left cheek | | |
| | | | | | medial, pectoral | | |
| | | | | | left, scapula left) | | |
| | | | | | actinic keratosis | | |
| | | | | | (flank left) | | |
| 02 | f | 82 | II | increased | squamous cell | Diclofenac | left forehead |
| | | | | exposure to | carcinoma (pectoral | 3% in 2.5% | (cf. FIG. 1/02) |
| | | | | holidays | left) | hyaluronic acid | |
| | | | | | M. Bowen (right | Photodynamic | |
| | | | | | abdomen, left | therapy | |
| | | | | | forehead, nose) | | |
| 03 | m | 77 | III | psoriasis vulgaris | melanoma (left | Diclofenac | dorsum left |
| | | | | et arthropathica | upper arm) | 3% in 2.5% | hand |
| | | | | (prior UV-therapy, | lentigo maligna | hyaluronic acid | |
| | | | | MTX 10 mg p.o.) | (right jaw angle) | 5% | |
| | | | | | basal cell | Imiquimod | |
| | | | | | carcinoma (nose) | | |
| | | | | | M. Bowen | | |
| | | | | | (dorsum right hand) | | |
| | | | | | actinic keratosis | | |
| | | | | | (2 x dorsum right | | |
| | | | | | hand) | | |
| 04 | f | 53 | II | extensive use of | actinic keratosis | Photodynamic | dorsum right |
| | | | | tanning beds | (>12, various | therapy | foot |
| | | | | father melanoma | locations) | 5% | (cf. FIG. 1/04) |
| | | | | mother actinic | squamous cell | Imiquimod | |
| | | | | keratosis | carcinoma (>12, | | |
| | | | | (capillitium, right | various locations) | | |
| | | | | malleolus | superficial | | |
| | | | | medialis, | spreading | | |
| | | | | Decolleté, dorsum | melanoma (gluteal | | |
| | | | | of both hands, | right) | | |
| | | | | both lower legs) | | | |
| | | | | daughter | | | |
| | | | | melanoma | | | |
| 05 | m | 75 | II | increased | — | Diclofenac | forehead |
| | | | | exposure in | | 3% in 2.5% | |
| | | | | childhood and | | hyaluronic acid | |
| | | | | holidays | | | |
| 06-1 | m | 77 | III | increased | basal cell | Diclofenac | left tibia |
| | | | | exposure in | carcinoma (>25, | 3% m 2.5% | |
| | | | | childhood and | various locations) | hyaluronic acid | |
| | | | | holidays | melanoma (upper | Cryotherapy | |
| | | | | | back) | 5% | |
| | | | | | | Imiquimod | |
| 06-2 | ″ | ″ | ″ | increased | basal cell | Diclofenac | left forehead |
| | | | | exposure in | carcinoma (>25, | 3% m 2.5% | (cf. FIG. 1/06-2) |
| | | | | childhood and | various locations) | hyaluronic acid | |
| | | | | holidays | melanoma (upper | Cryotherapy | |
| | | | | | back) | 5% | |
| | | | | | | Imiquimod | |
| 07 | m | 88 | II | — | basal cell | Diclofenac | right |
| | | | | | carcinoma | 3% in 2.5% | forehead |
| | | | | | (paravertebral left) | hyaluronic acid | |

TABLE 2
Lesion count and clinicai observations

| | | | | Total change | | |
|---|---|---|---|---|---|---|
| | Olsen | No. at | No. after | from baseline No. | | |
| Patient | Grade | baseline | treatment | (%) | | Clinical observations |
| 01 | I | 4 | 14 | +10 | (+250.00) | massive haemorrhagic crusts at baseline, |
| | II | 5 | 5 | 0 | (0.00) | decrease during treatments |
| | III | 19 | 1 | −18 | (−94.74) | (cf. FIG. 1/01) |
| | total | 28 | 20 | −8 | (−28.57) | |
| 02 | I | 1 | 4 | +3 | (+300.00) | decrease of erythematous maculae and |
| | II | 5 | 2 | −3 | (−60.00) | plaques |
| | III | 0 | 0 | 0 | (0.00) | (cf. FIG. 1/02) |
| | total | 6 | 6 | 0 | (0.00) | |
| 03 | I | 3 | 6 | +3 | (+100.00) | refined complexion |
| | II | 8 | 7 | −1 | (−12.50) | reduction of hyperpigmentation |
| | III | 2 | 0 | −2 | (−100.00) | reduction of hyperkeratosis |
| | total | 13 | 13 | 0 | (0.00) | |
| 04 | I | 8 | 22 | +14 | (+175.00) | reduction of hyperkeratosis |
| | II | 21 | 6 | −15 | (−71.43) | firmer complexion |
| | III | 3 | 0 | −3 | (−100.00) | (cf. FIG. 1/04) |
| | total | 32 | 28 | −4 | (−12.5) | |
| 05 | I | 2 | 7 | +5 | (+250.00) | reduction of hyperpigmentation |
| | II | 7 | 1 | −6 | (−85.71) | |
| | III | 0 | 0 | 0 | (0.00) | |
| | total | 9 | 8 | −1 | (−11.11) | |
| 06-1 | I | 2 | 2 | 0 | (0.00) | firmer skin |
| | II | 4 | 2 | −2 | (−50.00) | reduction of scaling |
| | III | 1 | 0 | −1 | (−100.00) | |
| | total | 7 | 4 | −3 | (−42.86) | |
| 06-2 | I | 2 | 4 | +2 | (+100.00) | reduction of scaling |
| | II | 13 | 4 | −9 | (−69.23) | (cf. FIG. 1/06-2) |
| | III | 0 | 0 | 0 | (0.00) | |
| | total | 15 | 8 | −7 | (−46.67) | |
| 07 | I | 2 | 1 | −1 | (−50.00) | refined complexion |
| | II | 3 | 0 | −3 | (−100.00) | reduction of hyperpigmentation |
| | III | 0 | 0 | 0 | (0.00) | reduction of scaling |
| | total | 5 | 1 | −4 | (−80.00) | |

I claim:

1. A method of treating actinic keratosis and/or non-melanoma skin cancer in a patient in need thereof, comprising exposing the affected area to cold atmospheric plasma for a time sufficient to induce an antitumoral response sufficient to destroy cancerous cells and/or repeating said application until clearance is achieved.

2. The method of claim 1, wherein the cold atmospheric plasma is an argon-containing plasma.

3. The method of claim 2, wherein the argon-containing plasma is generated by microwaves.

4. The method of claim 1, wherein the cold atmospheric plasma has a temperature of 20-100° C., preferably below 40° C.

5. The method of claim 1, wherein the affected area is exposed to cold atmospheric plasma for one to six sessions a week, preferably two sessions a week.

6. The method of claim 1, wherein the affected area is exposed to cold atmospheric plasma for one session a day, one session every two days, one session every three days, one session every four days, one session every five days or one session every six days.

7. The method of claim 1, wherein the affected area is exposed to cold atmospheric plasma for 10-300 seconds per session.

8. The method of claim 1, wherein the affected area is exposed to cold atmospheric plasma for 5-30 sessions in total.

9. The method of claim 1, wherein the patient is fair-skinned.

10. The method of claim 1, wherein the patient suffers from new or therapy-refractory actinic keratosis.

11. The method of claim 1, wherein the patient suffers from actinic keratosis Olsen grade I, II or III.

12. The method of claim 1, wherein the patient suffers from field-cancerization.

13. The method of claim 1, wherein the patient was previously treated with diclofenac.

14. The method of claim 1, wherein the patient was previously treated with imiquimod.

15. The method of claim 1, wherein the patient was previously treated with ingenol mebutate.

16. The method of claim 1, wherein the patient was previously treated with 5-fluorouracil or 5-fluorouracil with salicylic acid.

17. The method of claim 1, wherein the patient was previously treated with photodynamic therapy, preferably using (methyl)aminolevulinic acid or a salt thereof.

18. The method of claim 1, wherein the treatment comprises a simultaneous treatment with at least one active agent selected from diclofenac, ingenol mebutate, 5-fluorouracil or imiquimod or a simultaneous photodynamic therapy, preferably using (methyl)aminolevulinic acid or a salt thereof.

19. The method of claim 1, wherein the patient is not simultaneously treated with an additional active agent against actinic keratosis.

20. A method of treating actinic keratosis in a patient in need thereof comprising exposing the affected area to cold atmospheric plasma for a time sufficient to induce an antitumoral response sufficient to destroy cancerous cells and/or repeating said application until clearance is achieved.

21. The method of claim 19 wherein the cold atmospheric plasma application is for at least two minutes and is repeated once or twice a week until clearance is achieved.
